(19) United States
(12) Patent Application Publication
(10) Pub. No.: US 2004/0115777 A1
(43) Pub. Date: Jun. 17, 2004

(54) Identification of protein interactions using in vivo post-translationally modified fusion proteins

(75) Inventors: Paul Budworth, San Diego, CA (US); Xun Wang, San Diego, CA (US)

Correspondence Address: JENKINS & WILSON, PA, 3100 TOWER BLVD, SUITE 1400, DURHAM, NC, 27707, US

(73) Assignee: Syngenta Participations AG

(21) Appl. No.: 10/686,199

(22) Filed: Oct. 15, 2003

Related U.S. Application Data
(60) Provisional application No. 60/418,952, filed on Oct. 15, 2002.

(51) Int. Cl. G01N 33/53; C12P 21/04

(52) U.S. Cl. 435/69.7; 435/320.1; 435/325; 435/7.1

(57) ABSTRACT

Methods and products for the study of in vivo protein interactions and the identification of natural ligands or binding partners for proteins are disclosed. The methods employ fusion proteins comprising a protein of interest and a post-translational modification sequence which is post-translationally modified with a tag. The tag can then be used in an affinity purification methodology to extract both the fusion protein and its natural ligands or binding partners from a cell lysate. Also disclosed are vectors useful in producing such fusions, and cells transformed with such vectors.

### CROSS-REFERENCE TO RELATED APPLICATION

This application is based on and claims priority to U.S. Provisional Patent Application Serial No. 60/418,952, filed Oct. 15, 2002, the disclosure of which is incorporated herein by reference in its entirety.

### TECHNICAL FIELD

The presently disclosed subject matter relates to proteome analysis and the identification of protein interactions. More specifically, the presently disclosed subject matter relates to methods and products for identifying in vivo protein interactions using fusion proteins which are post-translationally modified in vivo to create tagged fusion proteins for affinity purification of complexes including the fusion protein.

### BACKGROUND ART

With the recent advancements in sequencing entire genomes, attention has turned to characterizing the function, modification, and regulation of the encoded proteins. Although studies of the function, modification and regulation of individual proteins have long been known in the art, several recent investigations have been devoted to ascertaining the biological function of proteins and their networks at a genome-wide or proteome-wide level (see e.g., Legrain et al., 2000; Zhu et al., 2001; Kumar et al., 2002; Tong et al., 2002).

The two-hybrid system in yeast is a system of choice to detect pair-wise protein-protein interactions via transcriptional activation of one or several reporter genes (Fields et al., 1989; Legrain et al., 2000). The system depends upon the creation of two fusion or hybrid proteins in which each hybrid protein includes one of a pair of activation domains which, when complexed, cause transcriptional activation of a reporter gene in yeast, but which have insufficient affinity for each other to maintain the complex. In the yeast two-hybrid system, the first activation domain is fused to a protein of interest which serves as a first binding domain, and the second activation domain is fused to a candidate protein which is tested for its ability to serve as a second binding domain that binds to the first binding domain. Binding between the protein of interest and the candidate protein can cause the activation domains to form the complex that causes transcriptional activation of the reporter. Thus, transcriptional activation of the reporter gene can be used as an assay for binding between the protein of interest and the candidate protein. Typically, the protein of interest is used as “bait” that is screened against a large library of candidate proteins. Using such a system, much progress has been made on genome-wide protein-protein interaction studies in the yeast Saccharomyces cerevisiae, largely due to the early publication of the Saccharomyces genome, and the practicality of the two-hybrid approach in this species (Uetz et al., 2000; Ito et al., 2000). The two-hybrid approach has also been applied to the systematic analysis of protein-protein interactions in the roundworm Caenorhabditis elegans (Walhout et al., 2000), mouse (Suzuki et al., 2001), and rice (Fang et al., 2002).

Because it is based on one-to-one protein interactions, the yeast two-hybrid system has several drawbacks when applied to large-scale high-throughput screening systems. First, if the protein of interest is a transcriptional activator, it may activate the reporter gene without any additional interacting proteins. Second, only two proteins are tested at a time, which means this method cannot identify components of a complex involved in a pathway that do not directly interact with the target protein. Third, it only predicts possible interactions, which may not represent what is happening under physiological conditions.

Rigaut et al., 1999 reported a strategy for protein complex characterization in which a “tandem affinity purification (TAP)” peptide tag was used to purify proteins associated or complexed with a fusion protein including the TAP tag. In this system, the TAP tag consisted of a calmodulin-binding domain (CBD) fused to a TEV cleavage site fused to a two Protein A IgG-binding units. This tag was then fused to a protein of interest (yeast U1 snRNP) and used to identify interacting proteins in yeast. After expressing the fusion protein in yeast, the tagged protein and any complexed binding partners were isolated from cell lysates using immobilized IgG. After washing away unbound material, the TEV sequence was cleaved, and the cleaved material was eluted. Finally, immobilized calmodulin was used to purify the tagged protein from the eluate. Although Rigaut et al., 1999 suggest that the TAP tag system has general application, published reports relating to the TAP tag and similar systems appear to be limited to studies in yeast (Rigaut et al., 1999; Gavin et al., 2002; Honey et al., 2001; Ho et al., 2002).

Biotin has also been used as an affinity tag to purify proteins. Biotin is an essential cofactor (vitamin H) for a set of enzymes involved in diverse metabolic processes, such as lipid metabolism, amino acid metabolism, and carbohydrate metabolism (Wang et al., 1994), and exists in many different organisms, including most bacteria, some fingi, plants and animals. For example, there are several biotin-containing enzymes reported in plants, including acetyl-CoA carboxylase, 3-methylcrotonyl-CoA carboxylase, propionyl-CoA carboxylase, pyruvate carboxylase and geranoyl-CoA carboxylase (Wang et al., 1994; Guan et al., 1999; Wurtele et al., 1990). Methylcrotonyl-CoA carboxylase (MCCase) is present in all plant organs and usually is the most prevalent of the biotin-containing enzymes. MCCase is composed of two non-identical subunits, a larger biotin-containing subunit MCC-A (˜85 kDa) and a smaller non-biotin-containing subunit MCC-B (˜60 kDa; McKean et al., 2000). Wang et al., 1994 isolated an MCC-A clone named TMC-B from tomato, and identified the conserved biotinylation site of the peptide at amino acid residues 399-402 of TMC-B. Wang et al., fused the C-terminal 70 amino acids (residues 367-436) peptide of the tomato TMC-B gene to beta-galactosidase and expressed the fusion protein in E. coli. The fusion protein was successfully biotinylated in E. coli and purified through affinity chromatography with immobilized avidin.

U.S. Patent No. 5,252,466 to Cronan describes a method of protein purification employing fusion proteins having a site for in vivo post-translation modification. Specifically, Cronan discloses fusion proteins in which a protein of interest is fused to a biotinylation site and the fusion proteins are biotinylated in vivo after the fusion proteins are expressed. The biotin is used as a tag to purify the fusion proteins. There is no discussion in Cronan, however, of using the biotin tags to identify natural ligands or binding partners of the protein of interest.

### SUMMARY

The presently disclosed subject matter depends, in part, upon the development of methods for the study of in vivo protein interactions and the identification of natural ligands or binding partners for proteins. The methods employ fusion proteins comprising a protein of interest and a sequence which is post-translationally modified to produce a tagged fusion protein. The tag can then be used in an affinity purification method to separate both the fusion protein and its natural ligands or binding partners from a cell extract.

Thus, in one aspect, provided is a method for obtaining in vivo binding partners of a protein of interest in a cell type. In one embodiment, the method comprises: (a) obtaining cells transformed to express a fusion protein of the protein of interest and a post-translational modification sequence heterologous to protein of interest; (b) growing the cells or progeny of the cells under conditions which permit expression and post-translation modification of the fusion protein to produce a tagged fusion protein; (c) contacting an extract of the cells or progeny of the cells with an affinity purification binding partner which specifically binds to the tagged fusion protein to form complexes; and (d) separating the complexes from the extract. The methods optionally can include the further step of identifying any binding partners of the protein of interest complexed to the protein of interest.

In a related aspect, provided is a method for obtaining in vivo binding partners of a protein of interest in a cell type comprising: (a) transforming a cell of the cell type with a vector encoding a fusion protein comprising a protein of interest and a post-translational modification sequence heterologous to protein of interest; (b) growing the cell or progeny of the cell under conditions which permit expression and post-translation modification of the fusion protein to produce a tagged fusion protein; (c) contacting an extract of the cells or progeny of the cells with an affinity purification binding partner which specifically binds to the tagged fusion protein to form complexes; and (d) separating the complexes from the extract. The methods optionally can include the further step of identifying any binding partners of the protein of interest complexed to the protein of interest.

In some embodiments, the disclosed fusion constructs optionally include a cleavage site which is interposed between the protein of interest and the post-translational modification sequence. After separating the complexes from the extract, the fusion protein can be cleaved and the portion including the protein of interest can be further separated from the portion including the tag.

In particular embodiments, the post-translational modification sequence is selected from a biotinylation, lipoylation, glycosylation, phosphorylation, methylation, sulfation, prenylation, acetylation, N-amidation, oxidation, hydroxylation, or myristylation sequence. Generally, the post-translational modification sequence can be any sequence which is post-translationally modified in the transformed cells to produce a tag which can be selectively recognized by an affinity binding partner.

In some embodiments, the cell type is a plant cell, such as cells from crop plants (e.g., corn, alfalfa, sunflower, canola, soybean, cotton, peanut, sorghum, wheat, tobacco), vegetables, ornamental plants and coniferous plants. In other embodiments, the cell type is an animal cell, such as nematode, insect, fish, amphibian, reptilian, avian or mammalian cells. In some embodiments, the mammalian cells are human, non-human primate, mouse, rat, hamster, cat, dog, pig, sheep or goat cells.

In some embodiments, the cells used in the disclosed methods are transformed with sequences which alter the post-translational modification abilities of the cells by encoding enzymes which are not normally expressed in the cells. In certain embodiments, the fusion protein can be the only protein expressed in the cells bearing the corresponding post-translational modification sequence and, therefore, the affinity purification of the fusion protein can be simplified and improved. In other embodiments, the cells are transformed with a vector encoding one or more post-translational modification enzymes which are naturally expressed in the cell. In these embodiments, increasing the level of expression of the post-translational modification enzymes increases the efficiency of the disclosed methods.

In other embodiments, the cells used in the disclosed methods are transformed with sequences encoding one or more heterologous proteins to determine whether the protein of interest interacts with the heterologous protein(s). In some embodiments, the cells are transformed with nucleic acids encoding a library of heterologous proteins which are screened for interactions with the protein of interest using the disclosed methods.

In another aspect, provided are genetic vectors including nucleic acid sequences encoding the fusion proteins.

In yet another aspect, the invention provides cells transformed with the genetic vectors of the invention.

### BRIEF DESCRIPTION OF THE DRAWING

The following drawing is illustrative of embodiments of the presently disclosed subject matter and is not meant to limit the scope of the presently disclosed subject matter.

FIG. 1 illustrates a fusion protein construct of the invention. FIG. 1 (A) shows the nucleic acid and corresponding amino acid sequences of the TEV-Biotin cassette. FIG. 1(B) is a schematic diagram of the pND05-TBP-Biotin genetic construct, in which the maize TBP cDNA is joined in-frame with the coding region of the TEV protease cleavage site and the 70 amino acid biotinylation sequence (“Biotin”) of the TMC-B clone of the tomato MCC-A gene.

### DETAILED DESCRIPTION

The patents and scientific and medical publications referred to herein establish knowledge that was available to those of ordinary skill in the art at the time the presently disclosed subject matter was made. The entire disclosures of the issued U.S. patents, published and pending patent applications, and other references cited herein are hereby incorporated by reference.

### I. Definitions

All technical and scientific terms used herein, unless otherwise defined below, are intended to have the same meaning as commonly understood by one of ordinary skill in the art; references to techniques employed herein are intended to refer to the techniques as commonly understood in the art, including variations on those techniques or substitutions of equivalent techniques which would be apparent to one of skill in the art. In order to more clearly and concisely describe the presently disclosed subject matter, the following definitions are provided for certain terms which are used in the specification and appended claims.

As used herein, the term “binding partner” means any of a pair of organic chemical moieties which, under physiological conditions, associate non-covalently to form a complex. Examples of binding partners include, without limitation, receptors and ligands, antigens and antibodies, enzymes and substrates, biotin and streptavidin, carbohydrates and lectins, and the like. Explicitly excluded from the meaning of binding partners are inorganic moieties such as water, gases, and ions.

As used herein, the term “fusion protein” means a protein having an amino acid sequence which is a sequential combination of the amino acid sequences of two or more other proteins. Thus, the N-terminal amino acid sequence of a fusion protein can correspond to the whole or partial amino acid sequence of one protein and the C-terminal amino acid sequence can correspond to the whole or partial amino acid sequence of another. In some instances, the internal sequences can correspond to the amino acid sequences of yet other proteins. In addition, the fused sequences can be joined by linker or spacer sequences which are non-naturally occurring or are based on other natural amino acid sequences.

As used herein, the term “post-translational modification” means any in vivo chemical alteration of a polypeptide or protein after the primary sequence of the protein has been translated. Post-translational modifications include, without limitation, biotinylation, lipoylation, glycosylation, and the like. The chemical moiety which is post-translationally added to the polypeptide or protein is sometimes referred to herein as a “tag”. Useful post-translational modifications include those which produce a tag for which there is a corresponding binding partner for affinity purification.

As used herein, the term “tagged fusion protein” means a fusion protein which has been post-translationally modified by the addition of a tag.

As used herein, the term “vector” means any genetic construct, such as a plasmid, phage, transposon, cosmid, chromosome, virus, virion, etc., which is capable transferring nucleic acids between cells. Vectors can be capable of one or more of replication, expression, and insertion or integration, but need not possess each of these capabilities. Thus, the term includes cloning, expression, homologous recombination, and knock-out vectors.

As used herein, the term “transforming” means introducing into a cell or an organism an exogenous nucleic acid or nucleic acid analog such that transient or stable expression of said nucleic acid or nucleic acid analog, or integration of said nucleic acid into the genome of cell or organism, is achieved. The term “transform” is used to embrace all of the various methods of introducing such nucleic acids or nucleic acid analogs, including, but not limited to the methods referred to in the art as transformation, transfection, transduction, or gene transfer, and including techniques such as microinjection, DEAE-dextran-mediated endocytosis, calcium phosphate co-precipitation, electroporation, liposome-mediated transfection, ballistic injection, particle-mediated delivery, viral-mediated transfection, and the like. Stably transformed cells are sometimes referred to as “transgenic” cells, and organisms comprising transgenic cells are referred to as “transgenic organisms”.

As used herein, the term “cleavage site” means any amino acid sequence in a protein which is subject to sequence-specific cleavage by a chemical or enzymatic reaction. Specifically excluded from the meaning of cleavage site are non-specific cleavage sites, such as the non-specific substrates of endopeptidases.

As used herein, the term “extract” means any preparation which is obtained from a cell and which includes a fusion protein as disclosed herein. In the case of fusion proteins which are secreted from cells grown in vitro, the extract can be the unrefined cell culture supernatant. In the case of fusion proteins which are membrane-bound (e.g., plasma, nuclear, endoplasmic reticulum, chloroplast, or mitochondrial membrane-bound), the cell extract can be the membrane fraction of a cell lysate. In the case of cytoplasmic or nucleoplasmic fusion proteins, the extract can be the non-membrane fraction of a lysate. As used herein, the term extract is not limited by the process by which the extract is obtained, and specifically includes, without limitation, such methods as chemical or enzymatic lysis, sonication, shearing or mechanical lysis, centrifugation and the like. The extract can be crude or highly purified as further described herein. The lysate can be obtained from cells grown in vitro, tissue culture, whole tissues or organs obtained from transformed or transgenic organisms, or whole organisms.

As used herein, the term “contacting,” as in the phrase “contacting A with B,” means that A and B are brought into sufficient physical proximity to interact at the molecular level, as by mixing A and B together in a solution, or pouring a solution of A over B on a substrate. As used herein, the phrase “contacting A with B” is intended to be equivalent to “contacting B with A” and is not intended to imply that either element is fixed relative to the other, or that either element is moved relative to the other.

As used herein, unless specifically indicated otherwise, the word “or” is used in the “inclusive” sense of “and/or” and not the “exclusive” sense of “either/or”.

### II. General Considerations

As noted above, the presently disclosed subject matter depends, in part, upon the development of methods for the study of in vivo protein interactions and the identification of binding partners for proteins. In the methods of the invention, fusion proteins are produced which include a protein of interest and a sequence which is post-translationally modified to produce a tagged fusion protein. The tag can then be used in an affinity purification method to separate both the fusion protein and its natural ligands or binding partners from a cell extract.

Rather than merely relying on a one-to-one protein interaction approach, as in the yeast two-hybrid system, the methods of the invention allow one to identify whole protein complexes interacting with a given target protein without prior knowledge of the complex composition, activity or function. In addition, rather than using affinity tags consisting of unmodified polypeptide sequences, as in the yeast two-hybrid system, the methods of the present invention employ affinity tags which are created in vivo by post-translational modification of the fusion protein. Moreover, in contrast to prior art uses of affinity tags merely to purify a fusion protein including a protein of interest, the presently disclosed methods employ affinity tags to identify natural ligands or binding partners of a protein of interest. Also, in contrast to prior methods of identifying protein interactions, the presently disclosed methods are shown to be useful in multicellular organisms, including plants and animals.

Thus, as described in the example below, provided herein is the first demonstration of in vivo protein interaction analysis in a multicellular organism, and also the first demonstration in which such a system has been successfully applied in the plant kingdom. In particular, an embodiment has been demonstrated in rice plants where each cell contains approximately 50,000 genes (Goff et al., (2002)). Despite the complexity of the rice genome and the large number of proteins found in each cell, only a single step of affinity purification was required to isolate six specific nuclei proteins which interacted with a protein of interest.

In one series of embodiments, the methods include the steps of: (a) obtaining cells transformed to express a fusion protein of the protein of interest and a post-translational modification sequence heterologous to protein of interest; (b) growing the cells or progeny of the cells under conditions which permit expression and post-translation modification of the fusion protein to produce a tagged fusion protein; (c) contacting an extract of the cells or progeny of the cells with an affinity purification binding partner which specifically binds to the tagged fusion protein to form complexes; and (d) separating the complexes from the extract. The methods optionally can include the further step of identifying any binding partners of the protein of interest complexed to the protein of interest.

In another series of embodiments, the methods include the steps of: (a) transforming a cell of the cell type with a vector encoding a fusion protein of a protein of interest and a post-translational modification sequence heterologous to protein of interest; (b) growing the cell or progeny of the cell under conditions which permit expression and post-translation modification of the fusion protein to produce a tagged fusion protein; (c) contacting an extract of the cells or progeny of the cells with an affinity purification binding partner which specifically binds to the tagged fusion protein to form complexes; and (d) separating the complexes from the extract. The methods optionally can include the further step of identifying any binding partners of the protein of interest complexed to the protein of interest.

The fusion constructs optionally include a cleavage site which is interposed between the protein of interest and the post-translational modification sequence. After separating the complexes from the extract, the fusion protein is cleaved and the portion including the protein of interest can be further separated from the portion including the tag.

### III. Fusion Constructs and Vectors

The fusion proteins of the present disclosure are based on essentially any protein of interest. For example, the protein of interest can be nuclear or cytoplasmic, membrane-bound or membrane-free, monomeric or multimeric. The protein need not be one which is normally post-translationally modified, and can be post-translationally modified in any manner which differs from the post-translational modification sequence of the fusion protein. However, both the protein of interest and the post-translational modification sequence should not be subject to the same post-translational modification.

The protein of interest need not be a complete or native protein. For example, isolated structural or functional domains of proteins, as well as other fragments, can be used. Similarly, naturally occurring or recombinantly produced mutant forms of proteins can be used as the protein of interest. In particular, naturally occurring post-translational modification sites can be removed from the protein by site-directed mutagenesis of the corresponding nucleic acid sequences in order to remove sites which would otherwise be subject to the same modification as the post-translational modification sequence of the fusion protein.

The fusion protein can comprise the protein of interest toward the N-terminus and the post-translational modification sequence toward the C-terminus or, conversely, the protein of interest toward the C-terminus and the post-translational modification sequence toward the N-terminus. The fusion protein can also include linker or cleavage site sequences between the protein of interest and the post-translational modification sequence. Moreover the fusion protein can include additional sequences which aid in protein expression or purification, such as leader sequences or additional affinity purification sequences (including, but not limited to polyhistidine, S. aureus protein A IgG binding domain, glutathione-S-transferase biding domain, maltose binding protein, cellulose binding domain, calmodulin binding peptide (Stratagene, La Jolla, Calif., United States of America), c-myc or other epitopes). Additionally, as a result of the techniques employed in producing the DNA encoding the fusion protein, there can be additional polypeptide sequences or “cloning artifacts” corresponding to portions of primer sequences, or restriction or ligation sites.

DNA or RNA encoding the fusion proteins can be produced according to any of numerous techniques well known to those of skill in the art. For example, nucleic acids can be synthesized de novo by chemical syntheses. Such techniques are particularly useful for the synthesis of short, artificial sequences. Alternatively, or in addition, the techniques of recombinant DNA technology can be used to isolate and manipulate nucleic acids produced by biosynthetic or chemical synthetic methodologies. The nucleic acids encoding the protein of interest can, for example, be obtained from mRNA, cDNA, or genomic DNA (gDNA) from cells expressing or encoding the protein, or from cDNA or gDNA libraries derived from such cells. Similarly, the post-translational modification sequences can be synthesized or isolated from a variety of sources.

The nucleic acids encoding the fusion proteins can be incorporated into any of a wide variety of vectors that can be used to transform the cell type to be studied. Such vectors can be expression vectors which allow transient expression of the fusion proteins, or integration or homologous recombination vectors which allow stable expression of the fusion proteins. The vectors can include various regulatory sequences, including operators, promoters, enhancers, ribosome binding sequences, termination sequences, polyadenylation signals and the like, which will aid in the expression of the fusion protein. The vectors can also include genetic elements necessary for the self-replication or integration of the vector, as well as selectable markers such as resistance and susceptibility genes. Methods of producing such fusion protein expression vectors are well known in the art and employ standard techniques of molecular cloning and recombinant DNA technology (see e.g., Sambrook & Russell, 2001. An example of the production of such a vector is described below.

### IV. Post-Translational Modification Sequences

The post-translational modification sequences disclosed herein can include any sequences which are post-translationally modified in such a way as to produce an affinity purification binding partner, and which encode a modification which is heterologous to the protein of interest. By “heterologous” to the protein of interest is meant that the post-translational modification is not naturally found in the protein of interest. Such sequences include biotinylation, lipoylation, glycosylation, phosphorylation, methylation, sulfation, prenylation, acetylation, N-amidation, oxidation, hydroxylation, or myristylation sequences. Generally, the post-translational modification sequence can be any sequence which is post-translationally modified in the transformed cells to produce a tag which can be selectively recognized by an affinity binding partner. In some embodiments, the post-translational modification is chosen to be one which is not common in the transformed cells.

Biotinylation sequences are present in a large variety of proteins from many different organisms, including bacteria, plants and animals. For example, there are several biotin-containing enzymes reported; in bacteria, including E. coli biotin carboxyl carrier protein (BCCP; a subunit of acetyl-CoA carboxylase) and the 1.3 S subunit of Propionibacterium shermanii transcarboxylase (PS 3S); in yeast, including S. cerevisiae pyruvate carboxylase (YPYC); in plants, including acetyl-CoA carboxylase, 3-methylcrotonyl-CoA carboxylase, propionyl-CoA carboxylase, pyruvate carboxylase and geranoyl-CoA carboxylase; and in humans, including human pyruvate carboxylase (HPYC).

Lipoylation sequences are present in proteins found in a variety of organisms, including bacterial species, such E. coli, B. stearothermophilus and A vinelandii; avian species, such as chickens; and mammalian species, such as rats, cows and humans. In post-translational lipoylation, lipoic acid is specifically added to a lysine residue in a lipoylation sequence by a lipoate ligase or lipoamide dehydrogenase (see, e.g., Stephens et al., (1983)).

Other post-translational modifications (e.g., glycosylation, phosphorylation, methylation, sulfation, prenylation, acetylation, N-amidation, oxidation, hydroxylation, or myristylation) are also known in the art and can be employed in the presently disclosed methods.

### V. Cleavage Sequences

Cleavage sequences optionally are included in the fusion proteins of the present disclosure, interposed between the protein of interest and the post-translational modification sequence. Such cleavage sequences can be used to further purify the protein of interest (and any associated binding partners) from the cell extract. For example, if the post-translational modification is one which occurs on a variety of other proteins within the cell, the affinity purification step will separate such proteins along with the fusion protein from the cell extract. In order to further purify the protein of interest, the fusion protein is cleaved at the cleavage site, and the remaining portion of the fusion protein is separated from the other proteins, which still bear the affinity tag.

Useful cleavage sites include the tobacco etch virus (TEV) specific cleavage sequence which is cleaved by the TEV protease NIA (Gibco BRL), the thrombin cleavage site, the papain cleavage site, and many others which are known to those of skill in the art. In addition to enzymatic cleavage sequences, chemical cleavage sites are also useful in the invention.

### VI. Transformed Cells

The presently disclosed methods are conducted with cells transformed to express the fusion proteins. Typically, the cell type is chosen based upon the identity and nature of the protein of interest. Thus, for example, if the protein of interest is a human protein, the fusion proteins can be expressed in human cells. Moreover, cell types characteristic of particular organs, tissues, or developmental states can be chosen to determine interactions of the protein of interest in such cells. Thus, for example, if the protein of interest is a plant protein involved in photosynthesis, the fusion protein can be expressed in leaf tissue.

Useful cell types include plant cells. A “plant” refers to any plant or part of a plant at any stage of development, including seeds, suspension cultures, embryos, meristematic regions, callus tissue, leaves, roots, shoots, gametophytes, sporophytes, pollen, and microspores, and progeny thereof. Also included are cuttings, and cell or tissue cultures. The term “plant cells” includes, but is not limited to, cells in whole plants, plant organs (e.g., leafs, stems, roots, shoots, leaves, meristems), differentiated and undifferentiated plant tissues, tumor tissues, plant seeds, pollen, protoplasts, embryos, callus tissue, and any groups of plant cells organized into structural and/or functional units, as well as plant cells grown in culture.

The methods of the invention can be employed to study protein interactions in a broad range of plant types, including the class of higher plants amenable to transformation techniques, particularly monocots and dicots. Useful monocots include species of the Family Gramineae including Sorghum bicolor and Zea mays. Other useful plants include species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, and Triticum.

Crop plants of particular interest include those from corn (Zea mays), canola (Brassica napus, Brassica rapa ssp.), alfalfa (Medicago sativa), rice (Oryza sativa). rye (Secale cereale), sorghum (Sorghum bicolor, Sorghum vulgare), sunflower (Helianthus annuus), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), duckweed (Lemna spp.), oats, and barley.

Vegetables of particular interest include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo).

Ornamental plants of interest include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbiapulcherrima), and chrysanthemum.

Coniferous plants of interest include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Isuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Alternatively, useful cell types are animal cells, such as nematode, insect (e.g., Drosophila), fish (e.g., zebrafish), amphibian (e.g., Xenopus), reptilian, avian, or mammalian cells. In particular, useful mammalian cells are human, non-human primate, mouse, rat, hamster, cat, dog, pig, sheep, or goat cells.

The cells can be obtained from cells grown in vitro, tissue culture, whole tissues, or organs obtained from transformed or transgenic organisms, or whole organisms.

The cells are transformed by any of the methods described herein or otherwise known in the art. Examples of methods for transformation of plants and plant cells include Agrobacterium-mediated transformation (De Blaere et al., 1985) and particle bombardment technology (Zhang et al., 1998; Chen et al., 1998; Klein et al., 1993; U.S. Pat. No. 4,945,050). Whole plants can be regenerated from transgenic cells by methods well known to the skilled artisan (see e.g., Fromm et al., 1990). Non-limiting examples of general methods of transformation for cells, including bacterial, fungi, plant and animal cells, include techniques such as microinjection, DEAE-dextran-mediated endocytosis, calcium phosphate co-precipitation, electroporation, liposome-mediated transfection, ballistic injection, particle-mediated delivery, viral-mediated transfection, and the like. Such methods are well known in the art.

The cells used in the presently disclosed methods can also be transformed with sequences which alter the post-translational modification abilities of the cells. Thus, for example, the cells can be transformed with genes encoding one or more post-translational modification enzymes (e.g., biotin ligase, lipoate ligase/lipoamide dehydrogenase, P-(1,4)-galactosyl transferase, prolyl 4-hydroxylase, gamma-glutamyl carboxylase, lysyl oxidase, lysyl hydroxylase, C-proteinase, N-proteinase, PACE, 7-glutamyl carboxylase, N-acetylglucosaminyl transferases, N-acetylgalactosaminyl transferases, sialyl transferases, fucosyl transferases, galactosyl transferases, mannosyl transferases, sulfotransferases, glycosidases, acetyl transferases, mannosidases) which are not normally expressed in the cells. See e.g., PCT International Publication No. WO 01/29242. By so doing, a wider range of post-translational modification sequences can be employed in the fusion proteins of the invention. In particular, if the cells are transformed with a post-translational modification enzyme which is not normally present in the cells, the fusion protein can be the only protein in the cells bearing the corresponding post-translational modification sequence and, therefore, the affinity purification of the fusion protein is simplified and improved. Alternatively, the cells can be transformed with a vector encoding one or more post-translational modification enzymes which are naturally expressed in the cell. By increasing the level of expression of these enzymes, however, the efficiency of the presently disclosed methods can be improved.

The cells used in the presently disclosed methods can also be transformed with sequences encoding one or more heterologous proteins to determine whether the protein of interest interacts with the heterologous protein(s). By “heterologous” is meant a protein not naturally produced in the cell or encoded by the genome of the cell. In some embodiments, the cells can be transformed with nucleic acids encoding a library of heterologous proteins which can be screened for interaction with the protein of interest using the presently disclosed methods.

### VII. Preparation of Extracts and Affinity Purification

The cell extracts can be essentially any preparation obtained from the transformed cells, tissues or whole organisms that includes the post-translationally modified fusion proteins and any associated binding partners. Thus, for fusion proteins secreted by cells grown in vitro, the cell extracts can be the unrefined supernatant from the cell culture or a highly purified fraction of the supernatant. For non-secreted fusion proteins, the extract can be a crude lysate of cells, tissues or organisms or a highly purified fraction obtained from the cells, tissues or organisms by any one or more of the many separation or purification techniques widely known in the art.

For example, crude cell lysates are prepared by lysing or disrupting cells by chemical, enzymatic, or mechanical approaches, including sonication or shearing (e.g., by French press). Cells also can be frozen and mechanically homogenized as described in the example below. Depending upon the nature of the fusion protein, a supernatant or crude lysate can be subjected to various separation or purification techniques to separate the fusion protein from other components of the cell culture medium or lysate. Such techniques include, without limitation, filtering, centrifugation, electrophoresis, chromatography, and dialysis. For membrane-bound proteins, such as plasma, nuclear, endoplasmic reticulum, chloroplast or mitochondrial membrane-bound proteins, the cell extract can be the membrane fraction of a cell lysate. Such fractions can be obtained by centrifugation of crude lysates. In the case of cytoplasmic or nucleoplasmic fusion proteins, the extract can be the non-membrane fraction of a lysate. Such membrane and non-membrane fractions can be obtained by centrifugation of a crude lysates. Proteins can be separated from large cellular structures by filtration, whereas small molecules can be removed by dialysis. Methods of electrophoresis and chromatography can be used to further separate proteins based upon size and electrical charge.

Prior to affinity purification using the post-translational tag, the extract can be subjected to affinity purification using one or more different affinity tags. For example, antibodies to undesired proteins present in the extract can be used to remove such proteins. Thus, if the post-translational modification of the fusion protein is lipoylation and it is known that several naturally-occurring proteins in the cells are lipoylated, antibodies to those proteins can be used to remove them from the extract prior to affinity purification using the lipoylation tag. Naturally, the antibodies should not be directed to the lipoylation moiety to avoid removing the lipoylated fusion protein.

### VIII. Identification of Binding Partners

After separating the complexes of the fusion proteins and affinity binding partners from the cell extract, the methods can include the additional step of identifying any binding partners of the protein of interest complexed to the protein of interest. These binding partners can be separated from the complexes by standard approaches, including elution with salt solutions, treatment with denaturing agents, and the like. The separated binding partners can then be subjected to standard means of physical and chemical analysis, including but not limited to, mass spectrometry, nuclear magnetic resonance spectroscopy, infra-red spectroscopy, electrophoresis, high performance liquid chromatography, tryptic digestion, protein sequencing, and the like. These and other methods of identifying unknown molecules are well known in the art.

### EXAMPLES

The following examples illustrate certain specific modes or embodiments of the presently disclosed subject matter, but are not intended to limit the scope of the presently disclosed subject matter. Alternative materials and methods may be utilized to obtain similar results.

### Example 1

### Identification of Binding Partners of Protein in Plants

As one exemplary embodiment, binding partners of a plant protein were identified. There is great interest in mapping protein-protein interactions in planta on a genome-wide scale. The recent release of the rice genome (Goff et al., 2002; Yu et al., 2002) highlights the increasing need to develop a system capable of utilizing such a database to study protein interactions in vivo on a multi-cellular level.

The TATA-box binding protein (TBP), which is well characterized in mammalian cells and known to be involved in the establishment of the transcription initiation complex (Zhu et al., 1995; Lemon et al., 2000; Naar et al., 2001; Sugiura, 1997; Orphanides et al., 2002; Lomvardas et al., 2001), was chosen as the test case protein of interest. A biotinylation sequence from tomato was used as the target sequence for in vivo post-translation modification and subsequent affinity purification. A fusion of the rice TBP protein and the tomato biotinylation sequence was transformed into rice cells grown in suspension. The biotinylated proteins were affinity purified from a whole protein extract and putative TBP binding partners were identified as described below.

### Fusion Constructs

Total RNA was prepared from mature tomato leaves using the RNeasy Maxi kit from Qiagen (Valencia, Calif., United States of America). This was used to synthesize cDNA with the SuperScript Choice System kit (Invitrogen Life Technologies, Carlsbad, Calif. United States of America). The sequence encoding the biotinylation sequence of the MCC-A biotin-containing subunit of tomato MCCase was cloned by PCR from tomato cDNA based on the sequences corresponding to nucleotides 1667-1880 of the TMC-B clone (Wang et al., 1994). The oligonucleotide primer sequences were:

(SEQ ID NO: 1)

5′CGGGATCCTTTCCCGGGGGTACTGTGATTGCACCCATGGC 3′
and

(SEQ ID NO: 2)

5′CTATCCGAGCTCTCAGTCCTTGAGAGCAAAGAGTTTTATAC 3′.

Restriction enzyme sites BamHI and XmaI were added in the 5′ primer, SacI was added in the 3′ primer, and are shown underlined above. The PCR product of the biotinylation sequence was cloned into a standard plant expression vector, designated pND0005, which contains the maize ubiquitin promoter and the NOS terminator. Two complementary oligos of the TEV protease cleavage site were synthesized (GENOSYS, Sigma Chemical Co., St. Louis, Mo., United States of America):

| | |
|---|---|
| 5′CGGGATCCAAAGGCCTACCGGTAAGATTCCAACTACTGCCAGCGAG 3′ | (SEQ ID NO: 3) |
| 5′AATTTGTATTTTCAGGGTGAGCTTAAAACCGCTCCCGGGGGTA 3′ | (SEQ ID NO: 4) |

The BamHI and StuI/AgeI restriction sites are underlined in SEQ ID NO: 3, and the XmaI site is underlined in SEQ ID NO: 4.

After annealing the oligos, the TEV cleavage site was excised by digestion with BamHI/XmaI and inserted into the BamHI/XmaI sites of the biotinylation sequence in pND0005, to give the construct pND05-Biotin. The multiple restriction sites (BamHI, StuI and AgeI) at the N-terminal of the TEV site are used for the cloning the gene for the protein of interest.

The maize TATA-box binding protein (TBP) gene was cloned from a maize full-length cDNA library using PCR amplification based on the GENBANK® database sequence (Accession No. L13301). The 5′ primer, with the BamHI site underlined, was 5° CGGGATCCATGGCGGAGCCGGGGCTCGAGG 3′ (SEQ ID NO: 5). The 3′ primer, with the AgeI site underlined, was 5′ GCGCACCGGTTTGCTGAACTTTTCGAAACTCTGCCAG) 3′ (SEQ ID NO: 6).

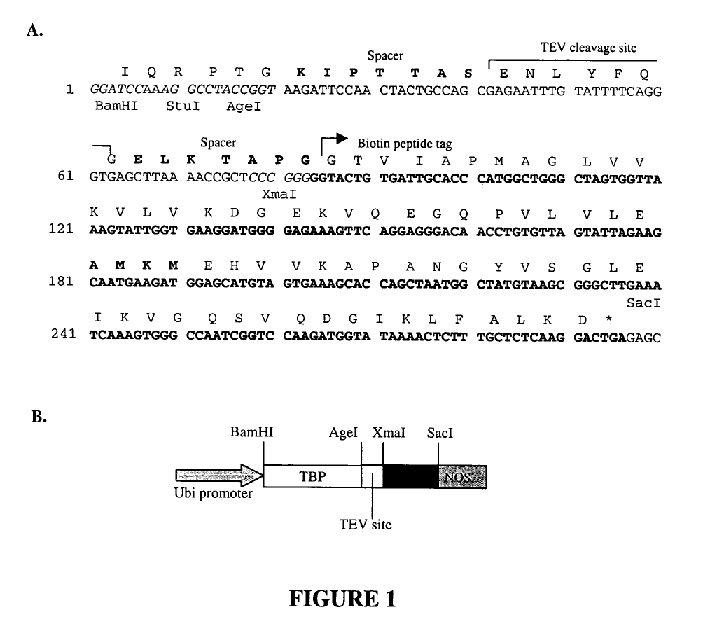

The TBP gene was inserted into the pND05-Biotin construct at the BamHI and AgeI sites to give the construct pND05-TBP-Biotin. As a result, the TBP gene was placed under the control of the maize ubiquitin promoter, and in-frame fused with the biotinylation sequence at the C-terminus, with the TEV cleavage site present as a linker region (FIG. 1(A)). Using a similar strategy, a single translation starting codon ATG was added at the AgeI site of pND05-Biotin, in-frame with the TEV-biotinylation sequence, to give an empty vector used as the control in the transformation. The DNA sequence was confirmed by sequencing. The protein sequence of maize TBP is 94% homologous with rice OsTBP2 (Zhu et al., (2002)).

### Rice Transformation and Transgenic Cell Maintenance

The pND05-TBP-Biotin construct and empty vector control were co-transformed with a plasmid, designated pCIB 7613 that contains the maize ubiquitin promoter driving expression of hygromycin (hpt). The rice suspension cells were derived from mature seeds of Oryza sativa L. japonica cultivar Taipei 309 and transformed by particle bombardment (Zhang et al., 1998; Chen et al., 1998). Stable transformants were selected on hygromycin-containing (50 mg/1) semi-solid media and then resuspended in liquid medium for large-scale culturing as previously described (Zhang et al., 1998).

### Protein Extract Preparation

Rice suspension cells were harvested and frozen in liquid nitrogen. Frozen tissues were homogenized to a fine powder with a mortar and a pestle under evaporating liquid nitrogen. The resultant powder was resuspended in 2 volumes of pre-chilled 10 mM potassium phosphate (pH 7.4), 50 mM NaCl, 5 mM EDTA, 1 mM PMSF, 0.5% Protease inhibitor cocktail (Sigma, St. Louis, Mo., United States of America). The mixture was filtered through 2 layers of Miracloth and the filtrates were centrifuged at 10,000 g for 20 min at 4° C. The supernatant was further filtered through a 0.22 μm Millipore membrane and the protein concentration was determined as described in Bradford, 1976 with the Bio-Rad protein assay reagents.

### Silver Staining and Western Blot Analysis

Protein extracts from a wild type line (wt) and four stable transgenic lines were grown on selection medium and prepared and checked for the TBP-Biotin fusion protein expression by using Western blot. Protein extracts were separated on 10% NuPAGE gels (Invitrogen, Carlsbad, Calif.) and stained using an Owl Silver Stain kit (Owl Separation Systems, Portsmouth, N.H.) according to the manufacturer's instructions. In order to detect the expression of biotin-tagged protein in the rice suspension cells, the separated protein bands were transferred from gels to nitrocellulose filters using a semidry transfer apparatus (Sambrook & Russell, 2001). The biotin-containing peptides were detected using Pierce ImmunoPure HRP-Streptavidin diluted at 1:50,000, and Pierce SuperSignal West Femto Maximum Sensitivity Substrate (Pierce Biotechnology, Inc., Rockford, Ill. United States of America).

### Purification of Biotin Tagged Protein

Fifty milligrams of protein extract were diluted to 45 ml in binding buffer (20 mM sodium phosphate pH 7.5, 100 mM NaCl) in a 50 ml Corning tube and incubated with 300 μl (3 mg) of 1% BSA-saturated MPG streptavidin (CPG Inc., Lincoln Park, N.J., United States of America) on a rotator at 4° C. overnight. The magnetic streptavidin coated beads and solution were then separated using a magnetic separator (Capture-Tec Stand from Invitrogen, Carlsbad, Calif., United States of America). After gently washing the beads 2-3 times with 45 ml each of binding buffer, the beads and bound proteins were incubated with 1.5 μl (10 units/μl) of 6X-His tagged TEV protease (Invitrogen Life Technology, Carlsbad, Calif., United States of America) in 300 μl of TEV cleavage buffer (50 mM Tris-Cl, pH 8.0, 0.5 mM EDTA, 1 mM DTT) on a rotator at 4° C. overnight. The cleavage solution containing TBP and its associated proteins was separated from the magnetic beads by placing the tube on the magnetic separator. The supernatant was collected and further incubated with 20 mM imidazole and 10 μl Ni-NTA magnetic agarose beads (Qiagen Inc., Valencia, Calif., United States of America) on a rotator for 1 hr at room temperature to remove the protease from the supernatant. The final purified protein supernatant was then collected using a magnetic separator, and used for further protein identification.

### Sample Preparation for LC-MS/MS Analysis

For 1-D electrophoresis, gels were run according to established methods using a BioRad mini-gel system and BioRad pre-cast gels. Protein bands from 1-D gels were visualized with silver staining, excised manually, and transferred to 96-well plates. The plates were transferred to a Massprep digestion robot (Micromass, Beverley, Mass., United States of America) for destaining (Gharahdaghi et al., 1999) and in-gel digestion with trypsin (Shevchenko, 1996)). Following digestion, tryptic peptides were extracted from the gel pieces with 5% formic acid/5% CH₃CN on the Massprep robot. The extracted peptides were diluted to 100 μl per well with 0.1% formic acid.

### High Performance Liquid Chromatography—Tandem Mass Spectrometry

A microbore HPLC system (Surveyor, ThermoFinnigan, San Jose, Calif., United States of America) was modified to operate at capillary flow rates using a simple T-piece flow-splitter. Columns (10 cm×75 μm I.D.) were prepared by packing 100 Å, 5 μm Zorbax C18 resin at 500 psi pressure into New Objectives Pico Frits (New Objectives, Mass., United States of America) columns with integral spray needles. Peptides were eluted in a gradient using buffer A (5% v/v acetonitrile, 0.1% formic acid) and buffer B (90% v/v acetonitrile, 0.1% formic acid), at a flow rate of 300 nl/min. Following an initial wash with buffer A for 10 minutes, peptides were eluted with a linear gradient from 0-100% buffer B over a 30 minute interval. Samples were introduced onto the analytical column using a Surveyor autosampler (Surveyor, ThermoFinnigan, San Jose, Calif., United States of America) which first transferred the 100 μl peptide extract onto a C 18 (300 μm×5 mm) cartridge (LC Packings, San Francisco, Calif., United States of America) and then used a switching valve to transfer the eluted peptides on to the analytical column. The HPLC column eluate was transferred directly into the electrospray ionization source of a ThermoFinnigan LCQ-Deca ion trap mass spectrometer (ThermoFinnigan, San Jose, Calif., United States of America). Spectra were scanned over the range 400-1400 mass units. Automated peak recognition, dynamic exclusion, and daughter ion scanning of the top two most intense ions were performed using the Xcalibur software as described previously (Haynes et al., 1998).

### Database Searching and Data Interpretation

MS/MS data were analyzed using SEQUEST, a computer program that allows the correlation of experimental data with theoretical spectra generated from known protein sequences (Yates et al., 1995). In this work, the criteria for a preliminary positive peptide identification for a doubly charged peptide were a correlation factor (Xcorr) greater than 2.5, a delta cross-correlation factor (ΔXcorr) greater than 0.1 (indicating a significant difference between the best match reported and the next best match), a minimum of one tryptic peptide terminus, and a high preliminary scoring. For triply charged peptides the correlation factor threshold was set at 3.5. All matched peptides were confirmed by visual examination of the spectra. All spectra were searched against a composite database containing a combination of a rice database and the non-redundant protein database (SWISSPROT). In cases where peptides were identified from unannotated sequence data, protein function was assigned where possible by BLAST homology searching (Altschul et al., 1990). SEQUEST result from every protein identified by three or fewer peptides were manually confirmed.

### TBP-Biotin Fusion Protein Expression in Rice Callus

The TBP gene was cloned from a maize full-length cDNA library, and the tomato biotinylation peptide was cloned from tomato leaf cDNA. FIG. 1(B) shows the final construct, pND05-TBP-Biotin, in which the maize TBP cDNA is in frame with the coding region of the TEV protease cleavage site and the 70 amino acid biotinylation sequence of the TMC-B clone of the tomato MCC-A gene. The pND05-TBP-Biotin construct was transformed into rice suspension cells by particle bombardment. Stable transgenic lines were chosen using the selection marker hygromycin. Western blotting with HRP-conjugated streptavidin identified two lines, B-3 and B-19, expressing the 32 kDa TBP-Biotin fusion protein. Line B-9 failed to express the transgene and line B-16 expressed truncated transgenes. The 85 kDa protein present in the wt and all of the transgenic lines is an endogenous biotinylated protein and is probably MCC-A, the most prevalent of the biotin-containing enzymes in plants. The detection of TBP-Biotin fusion protein in transgenic lines B-3 and B-19 with HRP-conjugated streptavidin demonstrated that the fusion protein was properly biotinylated in rice suspension cells. Lines B-3 and line B-19 were then chosen to be cultured in suspension on a large scale, and protein extracts were prepared from these culture for further analysis.

### Establishing Conditions for the Biotin-Tagged Protein Complex Purification

Since the TBP-Biotin fusion protein was properly biotinylated in rice suspension cells, the expressed fusion protein and its associated proteins were purified from rice suspension cell extracts under mild conditions by affinity chromatography with streptavidin coated magnetic beads. In order to determine the optimum ratios of streptavidin coated magnetic beads to protein extract, several different ratios of protein extract to Magnetic Porous Glass (MPG) streptavidin and several different incubation times were tested. Fifty milligrams of protein extract from lines B-3 and B-19 were tested separately for their binding capacity to 100, 200, 300, 400, or 500 μl of MPG streptavidin (10 mg/ml) by incubating the extracts with the resin on a rotator at 4° C. for either 2 hr or overnight. The complete protein extracts, post-binding supernatant, and streptavidin bound eluates were each analyzed by Western blot with HRP-conjugated streptavidin. Results showed that the majority of biotinylated proteins present in the protein extracts were bound to the beads when 50 mg of protein extract was incubated with 300 μl (3 mg) of MPG streptavidin at 4° C. overnight. These conditions were consequently used for all of the following biotin-tagged protein purification experiments. To identify the specific proteins associated with the TBP-Biotin expressing cell lines, 150 mg of protein extract from wt, B-3 and B-19 were prepared and incubated with 900 μl (9 mg) of MPG Streptavidin at 4° C. overnight. After gently washing the beads with binding buffer, we eluted the proteins bound to the beads using 6×His-tagged TEV protease. Following elution, the TEV-containing eluate (900 μl in total) was further incubated with 30 μl Ni-NTA magnetic agarose beads to remove the protease from the sample. A small aliquot (15 μl) of the TEV eluate from the wt and B-3 lines was tested for purification efficiency by running it on 10% NuPAGE gels and silver staining. Several bands were present in the transgenic line B-3, while only a few bands remained in the wt sample, indicating that non-specific binding had been virtually eliminated. In order to identify those specific bands isolated in the transgenic lines, all of the remaining eluate for each sample was concentrated and analyzed by LC-MS/MS.

### Mass Spectrometric Analysis of the TBP Complex

Replicate experiments of TBP-biotin purifications and analysis were preformed with either wild type callus or an empty vector control. In the second experiment, a clatherin protein was used as an additional control. The control line with empty vector (containing only the TEV-Biotin peptide) did not contain any significant protein bands. The TEV eluates from the TBP-biotin lines were analyzed on a gel and silver stained. TBP 1-D gel protein band patterns were nearly identical in both experiments. The excised protein bands were analyzed by LC-MS/MS. Proteins present in each of the bands were then identified by SEQUEST searching against a combination of a proprietary rice database and the NRP database from NCBI. SEQUEST results from the LC-MS/MS data showed most of the proteins identified to be the same in both experiments. Proteins identified from the clatherin-Biotin vector show very little overlap with the TBP-Biotin line. The results showed that most of the proteins identified were present in more than one band and with more than one peptide.

### Equivalents

While the presently disclosed subject matter has been particularly shown and described with references to certain embodiments thereof, it will be understood by those skilled in the art that various changes in form and details may be made therein without departing from the spirit and scope of the invention. Those skilled in the art will recognize, or be able to ascertain using no more than routine experimentation, many equivalents to the specific embodiments of the invention described herein. Such equivalents are intended to be encompassed within the scope of the invention.

### References

Altschul et al., (1990), “Basic local alignment search tool,” J Mol Biol 215, 403-410.

Bradford (1976), “A rapid and sensitive method for the quantification of microgram quantities of protein utilizing the principle of protein dye-binding,” Anal Biochem 72, 248-254.

Chen et al., (1998), “A protocol for consistent, large-scale production of fertile transgenic rice plants,” Plant Cell Reports 18, 25-31.

De Blaere et al., (1985), “Efficient octopine Ti plasmid-derived vectors for Agrobacterium-mediated gene transfer to plants”, Nucleic Acids Res 13(13):4777-88.

Fang et al., (2002), “Development of a high-throughput yeast two-hybrid screening system to study protein-protein interactions in plants,” Mol Genet Genomics 267, 142-153.

Fields et al., (1989), “A novel genetic system to detect protein-protein interactions,” Nature 340, 245-246.

Fromm et al., (1990), “Inheritance and expression of chimeric genes in the progeny of transgenic maize plants”, Biotechnology 8(9):833-9.

Gavin et al., (2002), “Functional organization of the yeast proteome by systematic analysis of protein complexes,” Nature 415, 141-147.

Gharahdaghi et al., (1999), “Mass spectrometric identification of proteins from silver-stained polyacrylamide gel: a method for the removal of silver ions to enhance sensitivity,” Electrophoresis 20, 601-605.

Goff et al., (2002), “A draft sequence of the rice genome (Oryza sativa L. ssp. japonica),” Science 296, 92-100.

Green (2000), “TBP-associated factors (TAFIIs): multiple, selective transcriptional mediators in common complexes,” Trends in Biochemical Sciences 25, 59-63.

Guan et al., (1999) “Geranoyl-CoA carboxylase: a novel biotin-containing enzyme in plants,” Arch Biochem Biophys 362, 12-21.

Hampsey (1998), “Molecular genetics of the RNA polymerase II general transcriptional machinery,” Microbiol Mol Biol Rev 62, 465-503.

Haynes et al., (1998), “Identification of gel-separated proteins by liquid chromatography-electrospray tandem mass spectrometry: comparison of methods and their limitations,” Electrophoresis 19, 939-945.

Ho et al., (2002), “Systematic identification of protein complexes in Saccharomyces cerevisiae by mass spectrometry,” Nature 415, 180-183.

Honey et al., (2001), “A novel multiple affinity purification tag and its use in identification of proteins associated with a cyclin-CDK complex,” Nucleic Acids Res 29, E24.

Ito, T. et al., (2000), “Toward a protein-protein interaction map of the budding yeast:

A comprehensive system to examine two-hybrid interactions in all possible combinations between the yeast proteins,” Proc Natl Acad Sci USA 97, 1143-1147.

Klein et al., (1993), “Particle bombardment: a universal approach for gene transfer to cells and tissues,” Curr Opin Biotechnol. 4(5):583-90.

Kumar et al., (2002), “Protein complexes take the bait,” Nature 415, 123-124.

Legrain et al., (2000), “Genome-wide protein interaction maps using two-hybrid systems,” FEBS Lett 480, 32-36.

Lemon et al., (2000) “Orchestrated response: a symphony of transcription factors for gene control,” Genes Dev 14, 2551-2569.

Lomvardas (2001), “Nucleosome sliding via TBP DNA binding in vivo,” Cell 106, 685-696.

McKean et al., (2000), “Molecular characterization of the non-biotin-containing subunit of 3-methylcrotonyl-CoA carboxylase,” J Biol Chem 275, 5582-5590.

Naar et al., (2001), “Transcriptional coactivator complexes,” Annu Rev Biochem 70, 475-501.

Orphanides (2000), “RNA polymerase II elongation through chromatin,” Nature 407, 471-475.

Orphanides (2002), “A unified theory of gene expression,” Cell 108, 439-451.

Pan et al., (2000), “Role of the TATA binding protein-transcription factor IIB interaction in supporting basal and activated transcription in plant cells,” Plant Cell 12, 125-136.

Proudfoot et al., (2002), “Integrating mRNA processing with transcription,” Cell 108, 501-512.

Rigaut et al., (1999), “A generic protein purification method for protein complex characterization and proteome exploration,” Nat Biotechnol 17, 1030-1032.

Sambrook & Russell, (2001), Molecular Cloning: A Laboratory Manual, Cold Spring Harbor Laboratory Press, New York.

Shevchenko et al., (1996), “Mass spectrometric sequencing of proteins silver-stained polyacrylamide gels,” Anal Chem 68, 850-858.

Stephens et al., (1983), “Nucleotide sequence of the lipoamide dehydrogenase gene of Escherichia coli K12”, Eur J Biochem 135(3):519-27.

Sugiura (1997), “Plant in vitro transcription systems,” Annu. Rev. Plant Physiol. Plant Mol. Biol 48, 383-398.

Suzuki et al, (2001), “Protein-protein interaction panel using mouse full-length cDNAs,” Genome Res 11, 1758-1765.

Tong et al., (2002), “A combined experimental and computational strategy to define protein interaction networks for peptide recognition modules,” Science 295, 321-324.

Uetz et al., (2000), “A comprehensive analysis of protein-protein interactions in Saccharomyces cerevisiae,” Nature 403, 623-627.

Von Mering et al., (2002), “Comparative assessment of large-scale data sets of protein-protein interactions,” Nature 417, 399-403.

Walhout et al., (2000), “Protein interaction mapping in C. elegans using proteins involved in vulval development,” Science 287, 116-122.

Wang et al., (1994), “Molecular cloning of cDNAs and genes coding for beta-methylcrotonyl-CoA carboxylase of tomato,” J Biol Chem 269, 11760-11768.

Wurtele et al., (1990), “Plants contain multiple biotin enzymes: discovery of 3-methylcrotonyl-CoA carboxylase, propionyl-CoA carboxylase and pyruvate carboxylase in the plant kingdom,” Arch Biochem Biophys 278, 179-186.

Yates et al., (1995), “Method to correlate tandem mass spectra of modified peptides to amino acid sequences in the protein database,” Anal Chem 67, 1426-1436.

Yu et al., (2002) “A draft sequence of the rice genome (Oryza sativa L. ssp. indica),” Science 296, 79-92.

Zhang et al., (1998), “Transgenic elite Indica rice varieties, resistant to Xanthomonas oryzae pv. oryzae,” Molecular Breeding 4, 551-558.

Zhu et al., (1995) “TATA box and initiator functions in the accurate transcription of a plant minimal promoter in vitro,” Plant Cell 7, 1681-1689.

Zhu et al., (2001), “Global analysis of protein activities using proteome chips,” Science 293, 2101-2105.

Zhu et al., (2002), “Rice TATA binding protein interacts functionally with transcription factor IIB and the RF2a bZIP transcriptional activator in an enhanced plant in vitro transcription system,” Plant Cell 14, 795-803.

What is claimed is:

1. A method for obtaining in vivo binding partners of a protein of interest in a cell type, the method comprising:
(a) obtaining a cell of said cell type transformed to express a fusion protein, said fusion protein comprising:
(i) said protein of interest; and
(ii) a post-translational modification sequence;
(b) growing said cell or progeny of said cell under conditions which permit expression and post-translation modification of said fusion protein to produce a tagged fusion protein;
(c) contacting an extract of said cell or progeny of said cell with an affinity purification reagent which specifically binds to said tagged fusion protein to form a complex; and
(d) separating said complex from said extract.

2. The method of claim 1, wherein an enzyme that performs the post-translation modification of said fusion protein is not naturally expressed in the cell of said cell type, and is encoded by a vector which is introduced into the cell.

3. The method of claim 1, wherein the cell is a mammalian cell.

4. The method of claim 1, further comprising identifying any binding partners of said protein of interest in said complexes.

5. The method of claim 1, wherein said fusion protein further comprises an affinity purification sequence selected from the group consisting of polyhistidine, S. aureus protein A IgG binding domain, glutathione-S-transferase binding domain, maltose binding protein, cellulose binding domain, calmodulin binding peptide, an epitope tag, and combinations thereof.

6. The method of claim 1, wherein said fusion protein further comprises a cleavage site between said protein of interest and said post-translational modification sequence, and said method further comprises the steps of:
(a) cleaving said cleavage site after separating said complex from said extract to form a cleaved protein of interest; and
(b) separating said cleaved protein of interest from said post-translational modification sequence prior to identifying any binding partners of said protein of interest.

7. The method of claim 1, wherein the protein of interest further comprises an affinity purification sequence selected from the group consisting of polyhistidine, S. aureus protein A IgG binding domain, glutathione-S-transferase binding domain, maltose binding protein, cellulose binding domain, calmodulin binding peptide, an epitope tag, and combinations thereof, and wherein the affinity purification sequence remains attached to the protein of interest after cleaving said cleavage site.

8. The method of claim 7, further comprising affinity purifying the protein of interest after the separating step.

9. A method for obtaining in vivo binding partners of a protein of interest in a cell type comprising:
(a) transforming a cell of said cell type with a vector encoding a fusion protein, the fusion protein comprising:
(i) said protein of interest;
(ii) a post-translational modification sequence;
(b) growing said cell or progeny of said cell under conditions which permit expression and post-translation modification of said fusion protein to produce a tagged fusion protein;
(c) contacting an extract of said cell or progeny of said cell with an affinity purification reagent which specifically binds to said tagged fusion protein to form a complex; and
(d) separating said complex from said extract.

10. The method of claim 9, wherein the cell is a mammalian cell.

11. The method of claim 9, further comprising identifying any binding partners of said protein of interest in said complexes.

12. The method of claim 9, wherein said fusion protein further comprises an affinity purification sequence selected from the group consisting of polyhistidine, S. aureus protein A IgG binding domain, glutathione-S-transferase binding domain, maltose binding protein, cellulose binding domain, calmodulin binding peptide, an epitope tag, and combinations thereof.

13. The method of claim 9, wherein said fusion protein further comprises a cleavage site between said protein of interest and said post-translational modification sequence, and said method further comprises the steps of:
(a) cleaving said cleavage site after separating said complex from said extract to form a cleaved protein of interest; and
(b) separating said cleaved protein of interest from said post-translational modification sequence prior to identifying any binding partners of said protein of interest.

14. The method of claim 9, wherein the protein of interest further comprises an affinity purification sequence selected from the group consisting of polyhistidine, S. aureus protein A IgG binding domain, glutathione-S-transferase binding domain, maltose binding protein, cellulose binding domain, calmodulin binding peptide, an epitope tag, and combinations thereof, and wherein the affinity purification sequence remains attached to the protein of interest after cleaving said cleavage site.

15. The method of claim 14, further comprising affinity purifying the protein of interest after the separating step.

16. A method for screening a plurality of potential binding partners for binding to a protein of interest, the method comprising:
(a) obtaining a cell transformed to express a fusion protein, said fusion protein comprising:
(i) said protein of interest; and
(ii) a post-translational modification sequence;
(b) growing said cell or progeny of said cell under conditions which permit expression and post-translation modification of said fusion protein to produce a tagged fusion protein;
(c) introducing into the cell the plurality of potential binding partners;
(d) contacting an extract of said cell or progeny of said cell with an affinity purification reagent which specifically binds to said tagged fusion protein to form a complex; and
(e) separating said complex from said extract.

17. The method of claim 16, wherein the plurality of potential binding partners are encoded by a plurality of nucleic acid expression vectors that when introduced into the cell result in the expression in the cell of the plurality of potential binding partners.

18. An expression vector encoding a fusion protein, wherein the fusion protein comprises a protein of interest and a post-translational modification sequence.

19. A cell comprising the expression vector of claim 18.


SEQUENCE LISTING (6 sequences)

SEQ ID NO: 1 - DNA, 40 bp, Artificial
  PCR primer used in conjunction with SEQ ID NO 2 to amplify a region of the tomato Methylcrotonyl-CoA carboxylase cDNA
cgggatcctttcccgggggtactgtgattgcacccatggc

SEQ ID NO: 2 - DNA, 41 bp, Artificial
  PCR primer used in conjunction with SEQ ID NO 1 to amplify a region of the tomato Methylcrotonyl-CoA carboxylase cDNA
ctatccgagctctcagtccttgagagcaaagagttttatac

SEQ ID NO: 3 - DNA, 46 bp, Artificial
  Complementary oligo A of the TEV protease cleavage site
cgggatccaaaggcctaccggtaagattccaactactgccagcgag

SEQ ID NO: 4 - DNA, 43 bp, Artificial
  Complementary oligo B to the TEV protease site
aatttgtattttcagggtgagcttaaaaccgctcccgggggta

SEQ ID NO: 5 - DNA, 30 bp, Artificial
  Oligo used in conjunction with SEQ ID NO 6 to clone the maize TATA-box binding protein
cgggatccatggcggagccggggctcgagg

SEQ ID NO: 6 - DNA, 37 bp, Artificial
  Oligo used in conjunction with SEQ ID NO 5 to clone the maize TATA-box binding protein
gcgcaccggtttgctgaacttttcgaaactctgccag